(19) United States
(12) Patent Application Publication
(10) Pub. No.: US 2019/0162665 A1
(43) Pub. Date: May 30, 2019

(54) ANALYSIS METHOD AND SYSTEM FOR ANALYZING A NUCLEIC ACID AMPLIFICATION REACTION

(71) Applicant: Roche Molecular Systems, Inc., Pleasanton, CA (US)

(72) Inventor: Rolf Knobel, Rotkreuz (CH)

(73) Assignee: Roche Molecular Systems, Inc., Pleasanton, CA (US)

(21) Appl. No.: 16/265,315

(22) Filed: Feb. 1, 2019

Related U.S. Application Data
(62) Division of application No. 14/834,262, filed on Aug. 24, 2015.

(30) Foreign Application Priority Data
Aug. 27, 2014 (EP) 14182415.1

(51) Int. Cl. G01N 21/64; C12Q 1/6851; G16B 45/00

(52) U.S. Cl. CPC G01N 21/6428; C12Q 1/6851; G01N 2021/6439; G01N 2201/12; G16B 45/00

(57) ABSTRACT

An analysis method is provided aimed at improving the linearity (precision and/or accuracy) of a quantification by a real-time nucleic acid amplification reaction RNR such as a polymerase chain reaction PCR over a wide range of analyte concentrations and/or limiting the effects of the presence of interfering substances by way of determining a quantification cycle number (Cq) of the RNR as the cycle number corresponding to an intersection of the growth curve with a combined threshold function (CTF) over the RNR cycle range comprising at least two different threshold levels, the quantification cycle number (Cq) being indicative of a quantitative and/or qualitative analysis result of a growth curve, the growth curve being indicative of the intensity of the fluorescence emission of an analyte for each amplification reaction cycle of the RNR over a RNR cycle range.

### CROSS-REFERENCES TO RELATED APPLICATIONS

The present application claims the benefit of priority under 35 U.S.C. § 119 of EP14182415.1, filed Aug. 27, 2014, the content of which is incorporated by reference herein in its entirety. This application is a divisional of U.S. application Ser. No. 14/834,262, filed Aug. 24, 2015, the disclosure of which is incorporated by reference in its entirety.

### FIELD OF THE INVENTION

The present disclosure relates to the field of analytics, and more particularly to an analysis method for a Real-time Nucleic acid amplification Reaction (RNR) such as a polymerase chain reaction PCR and a system for analyzing a nucleic acid amplification reaction of an analyte, such as for detecting a presence and/or measuring a quantity of an analyte in a sample by a nucleic acid amplification reaction.

### BACKGROUND OF THE INVENTION

In vitro nucleic acid amplification techniques include a variety of methods based on different approaches. One approach includes methods by which a DNA or RNA sequence is multiplied, thus making it more readily detectable for various procedures or tests. For example, in vitro amplification of the nudeic acid can be done using one of the following methods: Polymerase chain reaction (PCR), Ligase chain reaction (LCR), or isothermal transcription mediated amplification (TMA) method. In all of the above mentioned approaches the nudeic acid is subjected to repetitive amplification cycles.

A technique that is frequently used for various medical, biological or industrial applications is the PCR technique. This technique relies on thermal cycling, including cycles of repeated heating and cooling for enzymatic replication of the nucleic acid in order to amplify a specific region of the nucleic acid strand. As PCR progresses, the DNA generated is itself used as a template for replication, setting in motion a chain reaction in which the DNA template is exponentially amplified doubling (at least theoretically) with each reaction cycle.

Nucleic acid amplification techniques include many variations. One of these variations is called real-time nucleic acid RNR and is based on amplifying and simultaneously detecting the presence of nucleic acid in the sample and additionally to quantify its concentration in the sample.

There are several known methods for the detection of products in quantitative nucleic acid amplification. According to one of these methods, in order to indirectly measure the amount of nucleic acid during RNR, the intensity of a fluorescence emission of the analyte is acquired and a growth curve is created indicative of the intensity of the fluorescence emission over the RNR cycle range. The increase of the growth curve signal above the initially stationary baseline is used to determine a quantification cycle Cq in each reaction.

The determination of the quantification cycle Cq based on the growth curve using various CT methods is the subject of multiple patent applications:

EP 2719770 A1 discloses a method of detecting a presence and/or measuring a quantity of an analyte in a sample by PCR as well as a respective analyzer. In particular, signal normalization of the growth curve taking into account a maximum growth value of the signal is disclosed.

U.S. Pat. No. 7,788,039B2 discloses a method for determining an amount of target nucleic acid in a sample. An initial amount of the target is calculated according to a calibration equation using an initial amount of the standard, the target and standard growth curve values, where the calibration equation is a non-linear equation.

EP1798652B1 discloses a mathematical algorithm for creating a growth signal from measurement data.

EP1798542A1 shows a method for determining the presence of a nucleic acid in a sample wherein a digital value is selected on the growth signal and the presence of the nucleic acid is determined from a comparison of the selected digital value with calibrated digital values.

“Analyzing real-time PCR data by the comparative CT method”, Thomas D Schmittgen et al., Nature Protocols 3,—1101-1108 (2008) provides an overview of the comparative CT method for quantitative gene expression studies.

Nucleic acid amplification systems are commercially available from various vendors based on various nucleic acid amplification methods and employed for medical applications such as the detection of infectious diseases. Therefore, reliability and precision of nucleic acid amplification methods is of utmost importance.

Embodiments of the disclosed subject matter therefore aim to provide an improved analysis method using nucleic acid amplification and a respective system that enable improved robustness, precision, accuracy, linearity and qualitative discrimination of a nucleic acid amplification analysis.

### SUMMARY OF THE INVENTION

In accordance with embodiments of the disclosed subject matter, an analysis method for a real-time polymerase chain reaction is provided for detecting a presence and/or measuring a quantity of an analyte in a sample. Further, a system for analyzing a nucleic acid amplification reaction of an analyte is provided. Further embodiments are also provided.

Embodiments of the disclosed subject matter relate to an analysis method for a real-time nucleic acid amplification RNR using a combined threshold function CTF over the RNR cycle range, the CTF including at least two different threshold levels. The CTF is obtained by calculation on the basis of the at least two different threshold levels.

The calculation may be performed before or after the acquisition of the intensity of the fluorescence emission and the creation of the growth curve. For example, the calculation is performed once and the resultant CTF is stored in an electronic memory for reading out the CTF when it is needed for determining the quantification cycle number Cq of the RNR. Alternatively the CTF is calculated after the acquisition of the intensity of the fluorescent emission and/or after the creation of the growth curve before the determination of the quantification cycle number.

Embodiments of the disclosed subject matter are particularly advantageous as combining at least two different threshold levels into the CTF enables improvement of the precision of the RNR over a wide range of analyte concentrations and/or limit the effect of the presence of interfering substances.

Moreover, embodiments of the disclosed subject matter are particularly advantageous as the occurrence of false negatives for weak analyte concentrations and erroneous Cq values for high analyte concentrations are prevented thus increasing the reliable working range of the RNR analysis.

In accordance with embodiments of the disclosed subject matter at least one of the CT threshold levels is a constant value over the RNR cycle range. The constant value may be given by a relative intercept increase (RII-method), a partial growth threshold (PGT-method), a multiple of standard deviation of the growth curve above the baseline, a manually set threshold or a constant function.

In accordance with embodiments of the disclosed subject matter the CTF is obtained from the at least two different threshold levels by combining the threshold levels over the RNR cycle range, such as by superposition, linear or non-linear combination. Alternatively or in addition the CTF is obtained by dividing the RNR cycle range into RNR cycle intervals and calculating a separate CTF per interval using at least two different threshold levels per interval wherein the at least two different threshold levels can vary from interval to interval.

In accordance with an embodiment of the disclosed subject matter the CTF is a combination, such as a linear combination, of two threshold levels over the entire RNR cycle range. One of the threshold levels delivers a dominant contribution to the CTF at early reaction cycles of the RNR, such as for cycle numbers below 10, 15 or 20, whereas one of the threshold levels delivers a dominant contribution to the CTF at late reaction cycles of the RNR, such as for cycle numbers greater than 30, 40 or 50. In accordance with embodiments of the disclosed subject matter the CTF transitions from an initial threshold level to a final threshold level over the RNR cycle range. In other words, the contribution of the initial threshold level to the CTF may be dominant for early reaction cycles of the RNR such as for cycle number 0 or 1 and the contribution of the final threshold level to the CTF may be substantially below the contribution of the initial threshold level such as 0 at early reaction cycles of the RNR, such as for cycle number 0 or 1. Analogously, the contribution of the final threshold level at late reaction cycles of the RNR to the CTF may be substantially above the contribution of the initial threshold level. The transition of the CTF from the initial threshold level to the final threshold level over the RNR cycle range may be a step or multi-step transition, a linear transition, a polynomial transition, an exponential transition or a combination thereof.

In accordance with embodiments of the disclosed subject matter the initial threshold level itself comprises a first combination A of at least two different threshold levels. Alternatively or in addition, the final threshold level itself includes a second combination B of at least two different threshold levels where the threshold levels that are combined into the initial threshold level may be the same or different threshold levels than the threshold levels that are combined into the final threshold level.

In accordance with an embodiment of the disclosed subject matter one of the initial and final threshold levels is not a combination of threshold levels but a single threshold level.

In accordance with embodiments of the disclosed subject matter the initial threshold level comprises at least one threshold level that is adapted for precise determination of the quantification cycle number for a strong growth curve, i.e., a growth curve obtained for a high analyte concentration and/or low concentration of an interference substance.

On the other hand, the final threshold level includes at least one threshold level that is adapted for precise determination of the quantification cycle number for late increases, i.e., a low analyte concentration and/or high concentration of an interference substance. Due to the transition of the CTF from the initial threshold level to the final threshold level over the RNR cycle range a precise determination of the quantification cycle number is enabled both for early and late signal increases.

In accordance with embodiments of the disclosed subject matter the initial threshold level has a starting point with a y-coordinate below a y-coordinate of an end point of the final threshold level resulting in an increasing combined threshold function with a positive slope or the initial threshold level has a starting point with a y-coordinate above a y-coordinate of an end point of the final threshold level resulting in a decreasing combined threshold function, i.e. in a combined threshold function that has a negative slope over the RNR cycle range.

In accordance with embodiments of the disclosed subject matter the y-coordinate of the starting point and the y-coordinate of the end point are used to calculate the combined threshold function by a linear, polynomial, exponential, logarithmic or other transition between the two y-coordinates. For example, the two y-coordinates are stored in an electronic memory and read from the electronic memory for calculating the combined threshold function by means of linear interpolation between the two y-coordinates.

In accordance with embodiments of the disclosed subject matter the CTF is a constant over the RNR cycle range. For example each of the threshold levels provides a constant and the combined threshold function is calculated as a linear combination of the two constants such as an average or a weighted average of the two constants. For example, one of the threshold levels is adapted for precise determination of the quantification cycle number for an early signal increase whereas the other of the two threshold levels may be adapted for precise determination of the quantification cycle number for a late signal increase hence for CT determination at late reaction cycles of the RNR.

In accordance with another aspect of the disclosed subject matter a system for analyzing a nucleic acid amplification reaction of an analyte is provided, the system comprising: a detection system configured to acquire the intensity of fluorescence emission of the analyte for each amplification reaction cycle of the RNR and a processing unit configured to: create a growth curve indicative of the intensity of the fluorescent emission over a range of reaction cycles of the RNR, provide a combined threshold function CTF comprising at least two different threshold levels; and determine a quantification cycle number Cq of the RNR, the quantification cycle number Cq being indicative of a quantitative and/or qualitative analysis result of the growth curve as the cycle number corresponding to an intersection of the growth curve with the combined threshold function.

In accordance with embodiments of the disclosed subject matter the processing unit provides the combined threshold function by reading data from an electronic memory of the system that is descriptive of the combined threshold function and calculating the combined threshold function using this data and/or the growth curve. For example, the data stored in the electronic memory indicates the y-coordinate of the starting point of the initial threshold level and the end point of the final threshold level and the calculation of the combined threshold function by the processor is performed by means of an interpolation between the y-coordinates over the RNR cycle range.

Embodiments of the disclosed subject matter may be particularly advantageous as the accuracy and precision of the analysis may be increased over a wide concentration range of the analyte and/or robustness against interferences.

### BRIEF DESCRIPTION OF THE FIGURES

Other and further objects, features and advantages of the embodiments will appear more fully from the following description. The accompanying drawings, together with the general description given above and the detailed description given below, serve to explain the principles of the embodiments.

FIG. 1 shows a block diagram illustrating a schematic of a RNR analyzer for analyzing a sample, such as a biological sample.

FIG. 2 shows a schematic illustration of a RNR growth curve of an analyte for each RNR cycle.

FIG. 3A shows a schematic illustration of two different RNR growth curves, two threshold levels and a resultant CTF.

FIG. 3B shows a schematic illustration of two different RNR growth curves, two threshold levels and a resultant CTF.

FIG. 4 shows a schematic illustration of an alternative CTF for the growth curves of FIG. 3.

FIG. 5 shows a schematic illustration of a window that is displayed by an analyzer with a negatively sloped CTF.

FIG. 6 shows a schematic illustration of a window that is displayed by an analyzer with a negatively sloped CTF, the CTF transitioning from RII as initial threshold level to PGT as final threshold level.

FIG. 7 shows a schematic illustration of a window that is displayed by an analyzer with a positively sloped CTF.

FIG. 8 shows a schematic illustration of examples for linear, quadratic and exponential transition between the initial and final threshold levels.

FIG. 9 shows a schematic illustration of various growth curves of a dilution series.

FIG. 10 shows a schematic illustration of a comparison of various Cq values obtained using known threshold levels and the inventive CTF.

FIG. 11 shows a schematic illustration of various calibration curves of Cq values as a function of concentration for known threshold levels and the inventive CTF.

FIG. 12 shows a schematic illustration of growth curves of interferences.

FIG. 13 shows a schematic illustration of thresholds of interferences.

FIG. 14 shows a schematic illustration of Cq values as function of interference growth curve.

### DETAILED DESCRIPTION OF THE INVENTION

By way of illustration, specific exemplary embodiments in which the disclosed subject matter may be practiced now are described.

Certain terms will be used in this patent application, the formulation of which should not be interpreted to be limited by the specific term chosen, but as to relate to the general concept behind the specific term.

The term ‘baseline’ refers to the initial portion of the growth curve which shows no signal increase.

The term ‘intercept’ or ‘intercept value’ as understood herein is the signal offset value of the growth curve at an early cycle number, i.e., 0 that is obtained by extrapolation.

The term ‘saturation line’ refers to the portion of the growth curve in the plateau region after the exponential growth phase which is also referred to as exponential amplification phase and after the leveling off stage.

The term ‘threshold level’ as used herein encompasses a threshold level for the growth curve.

A ‘combined threshold function’ or ‘CTF’ as understood herein encompasses a mathematical function over the cycle range that is composed of at least two different threshold levels. According to one embodiment, a CTF may be a linear or logarithmic combination of two different threshold levels.

In one embodiment, the Y-value, i.e. the threshold level, returned by the CTF for a given X-value, i.e., cycle number, may transition from one of the two different threshold levels to the other one.

According to one embodiment, a CTF may transition from a first combination (e.g. linear or logarithmic) of at least two different threshold levels to a second combination (e.g. linear or logarithmic) of at least two different threshold levels.

The term ‘real-time nucleic acid amplification RNR cycle range’ as understood herein refers to the range of cycle numbers that is executed for acquiring the intensity values for the creation of the growth curve. For example, the RNR cycle range may be a predefined fixed number, such as 30, 40 or 50. Setting the RNR cycle range is a tradeoff between throughput of the analysis system for analyzing the nucleic acid amplification reaction and the sensitivity of the analysis system for the determination of a quantification cycle number for weak signals.

In accordance with an embodiment of the disclosed subject matter a threshold method is implemented as a relative intercept increase RII. For example, the RII is a parallel above the baseline.

In accordance with embodiments of the disclosed subject matter the RII may have the following format:

y(x)=B+r·I
B: baseline function, typically linear
I: intercept extrapolation to cycle number 0
r: positive percentage parameter, e.g. 50%

In accordance with embodiments of the disclosed subject matter a threshold method may be implemented as a partial growth threshold PGT that depends on the signal difference of the growth curve between the saturation line and the baseline, i.e. the growth from the baseline to the saturation line. The PGT may return a constant Y-value over the entire range of cycle numbers.

In accordance with embodiments of the disclosed subject matter the PGT may have the following form:

y(x)=B+p·G=B+p·(S−B) (2)
B: baseline function
S: Saturation line
G: growth from baseline to saturation line
p: parameter preferably between 0% and 100%, e.g. 5%

In accordance with an embodiment of the disclosed subject matter a threshold method is given by the standard deviation of the growth curve above the baseline and may have the following form:

y(x)=B+f·D (3)
B: baseline function
f: Multiplying factor parameter, e.g. 10
D: Standard deviation of growth curve

In accordance with embodiments of the disclosed subject matter a threshold method is given by a threshold value that is pre-set or manually entered by a user.

In accordance with an embodiment of the disclosed subject matter the threshold method is a constant value that is constant over the RNR cycle range, for example:

y(x)=C (4)
C: a predefined constant based on empirical data.

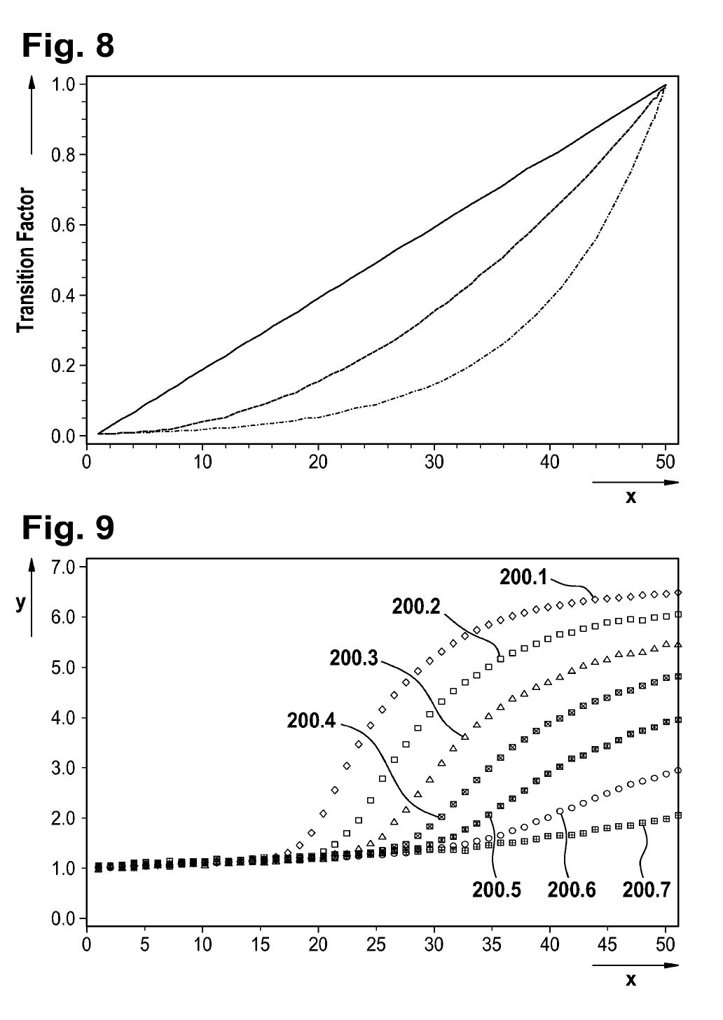

In accordance with embodiments of the disclosed subject matter the transition from one of the at least two different threshold levels, i.e. the initial threshold level, to the other one of the at least two different threshold levels, i.e. the final threshold level, is such by a step or multi-step, linear, polynomial and/or exponential transition as illustrated on FIG. 8.

For example, a CTF with a linear transition from the initial threshold level y₁ to the final threshold level y₂ may have the following form:

$$CTF ( x ) = {y}_{1} + ( {y}_{2} - {y}_{1} ) \cdot \frac{x - {x}_{1}}{{x}_{2} - {x}_{1}} \qquad ( 5 )$$

where:
x₁: early reaction cycle number, such as 0 or 1;
x₂: late reaction cycle number, such as 30, 40 or 50;
y₁: initial threshold level;
y₂: final threshold level.

In accordance with an embodiment of the disclosed subject matter the CTF may have the following format for implementation of a polynomial transition:

$$CTF ( x ) = {y}_{1} + ( {y}_{2} - {y}_{1} ) \cdot {( \frac{x - {x}_{1}}{{x}_{2} - {x}_{1}} )}^{p} \qquad ( 6 )$$

where:

p: Fixed polynomial order, such as 2 for a quadratic polynomial function.

In accordance with an embodiment of the disclosed subject matter the CTF may have the following format for implementation of an exponential transition:

$$CTF ( x ) = {e}^{\frac{{w}_{2} \cdot ln ( {y}_{2} ) - {w}_{1} \cdot ln ( {y}_{2} )}{{w}_{2} - {w}_{1}}} \cdot {( \frac{{y}_{2}}{{y}_{1}} )}^{\frac{w}{{x}_{2} - {x}_{1}}} \qquad ( 7 )$$

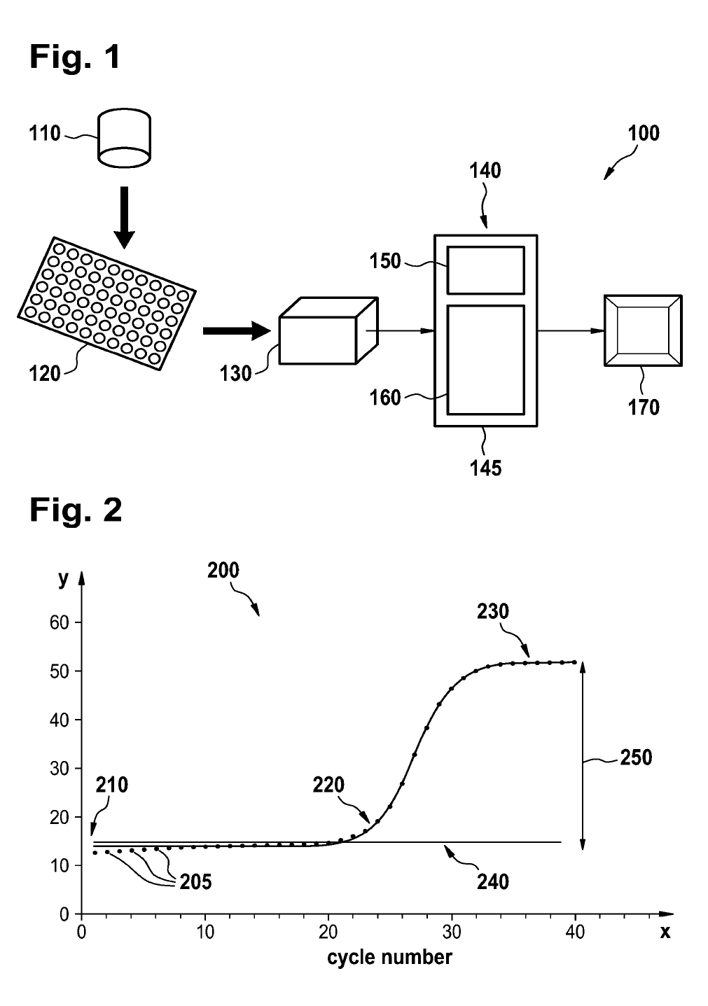

FIG. 1 shows schematics of the RNR system 100 for amplification and simultaneous quantification of an analyte in real time. Using RNR method, a signal is created and detected during the amplification process. The signal generally represents the amount of any analyte created during amplification and thus present in the sample. The sample is a liquid that may contain the analyte and/or other products of the amplification reaction. The RNR system comprise a thermal cycler block 120, an excitation light source 110, a detector system 130 for collecting the RNR signal in real time, and a data processing system 140 comprising a processing unit and a memory 150 for storing the RNR signal and program instructions 160 for analyzing the RNR signal, and a unit 170 for displaying the signal and outputting a result of the analysis.

The analyte is conjugated to a fluorescent dye and the sample is loaded into the thermal cycler block 120. A thermal cycler block 120 can be a conventional design sample block, which comprise 96 wells and is able to hold up to 96 samples. The sample is illuminated with the fluorescence excitation source 110, and the raw fluorescence data is measured by the RNR detector system 130 for each RNR cycle number. The RNR detector 130 is suitable to collect the RNR fluorescence signal emitted by one or more fluorescent dyes. The measured data is collected in data processing system memory unit 150, and can be displayed on the display unit 170 as an un-normalized RNR growth curve, or alternatively as a normalized RNR growth curve.

FIG. 2 shows the schematic example of a growth curve 200 representing the RNR signal taken for each RNR cycle. The diagram includes a Cartesian coordinate system where the abscissa is designated as the x-axis and the ordinate is designated as the y-axis and where x is the cycle number of the RNR and y is the intensity of the fluorescent emission.

FIG. 2 also shows fluorescence intensity values 205 as a dotted line, where each of the intensity values 205 has been acquired by performing a fluorescence emission intensity measurement of the ongoing amplification reaction. The intensity values 205 are modeled using a mathematical growth curve model formula or another kind of interpolation and/or interpolation. The RNR signal is showing as growth curve 200 in FIG. 2. Here the intercept value 210 is the RNR signal offset value when the RNR cycle number is zero.

The baseline 240 is the RNR signal during the initial cycles of the RNR reaction, typically measured between cycles 1 and 15, where there is no detectable increase in fluorescence due to RNR reaction products. The baseline 240 is the baseline function B (cf. equations 1 to 3). The pre-defined threshold 220 is used to determine a cycle number at which the RNR signal exceeds the baseline 240 of the RNR reaction, i.e. the cycle in which there is the first detectable significant increase in fluorescence, which is about c_{q}=22 here. The threshold 220 is given by CTF (cf. equations 5 to 7).

The maximum growth value 250 is a difference between a maximum intensity of the RNR signal at a plateau region 230 and the RNR signal at the baseline 240. The plateau stage 230 is the RNR signal during final cycles of the RNR reaction.

The intensity of the RNR signal at a plateau region 230 is S and the maximum growth value 250 is the growth G in the above equation 2.

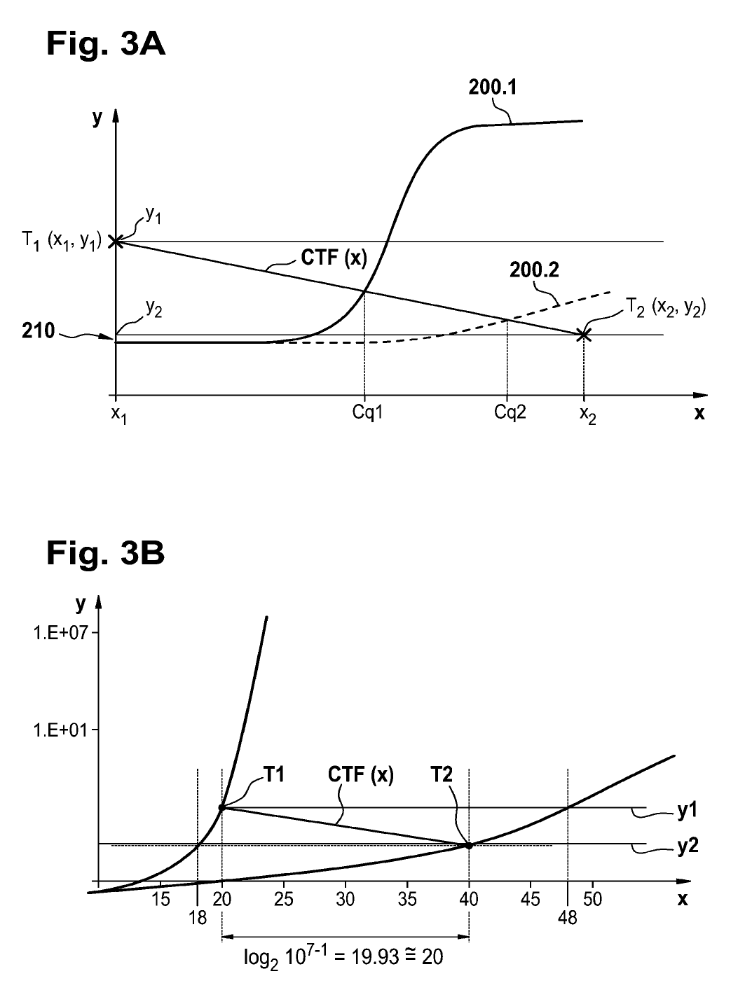

FIG. 3A shows a diagram with a graphical representation of a growth curve 200.1 and another growth curve 200.2 which is shown as a dashed line. The diagram includes a Cartesian coordinate system where the abscissa is designated as the x-axis and the ordinate is designated as the y-axis analogous to the diagram shown in FIG. 2 where x is the cycle number of the RNR and y is the intensity of the fluorescent emission.

The diagram shows a first threshold level y1 and a second threshold level y2.

The threshold y1 may be defined by entering a starting point T₁ (x₁, y₁); likewise the threshold level y2 may be defined by entering an end point T₂ (x₂, y₂).

In the embodiment considered here the y-coordinate of the starting point T₁ that determines the threshold level y1 is greater than the respective coordinate of y₂ of the end point T₂ such that y1 is above the other threshold level y2 as illustrated in FIG. 3A.

The growth curve 200.1 originates from a sample concentration at the upper end of the dynamic range of the RNR whereas the sample concentration for the growth curve 200.2 is at the lower end of the dynamic range. As a consequence, the exponential phase of the growth curve 200.1 occurs in a much lower cycle number range that is the case for the growth curve 200.2 as illustrated in FIG. 3A.

Application of the threshold level y1 alone for the determination of c_{q} of growth curves 200.1 and 200.2 would result in the detection of c_{q} only for growth curve 200.1 but would fail to detect a c_{q} value (or lead to very late detection) for growth curve 200.2 as the latter is too weak to reach the intensity y₁ in its exponential phase and/or plateau region within the RNR cycle range.

On the other hand, usage of threshold level y2 alone would lead to the detection of a c_{q} value for growth curve 200.2 but would be unreliable as regards detection for a c_{q} value for growth curve 200.1 as y₂ is close to the baseline such that variations could lead to an imprecise C_{q} detection for the growth curve 200.1.

This situation is remedied by combining threshold levels y1 and y2 which provides a combined threshold function CTF(x). In the example considered here, the CTF is in accordance with above equation 5 such that CTF has a linear transition from y₁ to y₂ over the x-coordinate range x₁ to x₂ as illustrated in FIG. 3A. This has the beneficial effect that the CTF intersects with both growth curves 200.1 and 200.2 within their respective exponential growth phases resulting in a reliable and precise detection of the respective quantification cycle numbers c_{q1} and c_{q2}.

Hence, the combined threshold function CTF(x) is calculated by combining the threshold levels y1 and y2. In the embodiment considered here this is a linear combination providing a linear transition from T₁ to T₂ along the x-axis. The RNR cycle range can be set to be equal to the x-coordinate range x₁ to x₂ in the embodiment considered here as additional RNR cycles above cycle x₂ would not provide a significant contribution to the quantification cycle number determination but merely reduce system throughput.

The calculation of the CTF can be performed by the RNR system 100 (cf. FIG. 1). For example, the coordinates of the points T₁ to T₂ are stored in the memory 150. By execution of the program instructions 160 T₁ to T₂ are read from the memory 150 and the combined threshold function CTF is calculated such as in accordance with equation 5, 6 or 7. Alternatively the combined threshold function CTF is stored in the memory 150 by means of data that is descriptive of the CTF, such as in tabular form.

In the example illustrated in FIG. 3A the CTF transitions from the initial threshold level y1 to the final threshold level y2. In the example considered here the transition is linear and results in a negatively sloped CTF whereby the initial threshold level y1 provides the initial starting point T₁ at cycle number x₁=0 and the final threshold level y2 provides the end point T₂ of the CTF at cycle number x₂.

FIG. 3B is illustrative of an alternative choice of the levels y1 and y2. In the example considered here usage of threshold level y1 alone would not result in a false negative as it would be the case for the FIG. 3 example. However, the point of intersection of the growth curve 200.2 with the CTF threshold level y1 is only at a late cycle number C_{q2}, i.e. at x=48 instead of x=40 (i.e. ca. log₂ 10⁶=19.93≅20 cycles later than C_{q1} corresponding to a ratio of 10⁶ between the concentration of the first respectively second analyte). This is disadvantageous as a relatively large number of cycles have to be executed for a qualitative result reducing throughput of the analysis system. Another disadvantage is that the point of intersection between CT threshold level y1 and the growth curve 200.2 may occur after the exponential growth phase of the growth curve 200.2 where the RNR process is in its leveling off stage (i.e. the amount of nucleic acid is no longer doubled at every cycle) leading to false C_{q} values. This situation is improved by using the CTF that results from the combination of y1 and y2 as the point of intersection and hence C_{q} for growth curve 200.2 is moved to x=40.

Alternatively CTF(x) can be in accordance with above equation 6 or 7 or it can be another monotonous or step-function.

In accordance with embodiments of the disclosed subject matter the threshold level y1 is in accordance with equation 2 thus taking into account the signal level S of the saturation line in the plateau phase whereas the threshold level y2 is in accordance with equation 1 taking into account the intercept value I rather than S.

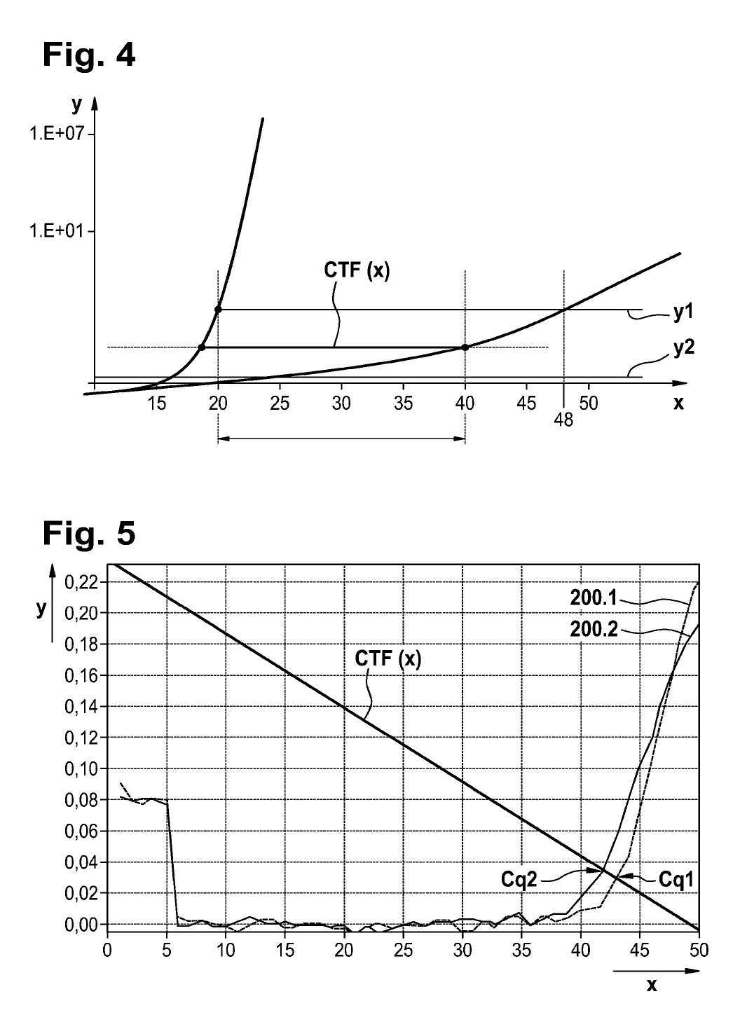

FIG. 4 illustrates an alternative embodiment for the combination of the thresholds y₁ and y₂ by means of a linear combination, such as CTF (x)=0.5 y1+0.5 y2 which is a constant and thus there is no transition of CTF in this case, wherein the threshold level y1 is adequate for early reaction cycles of the RNR while the threshold level y2 being adequate for late reaction cycles of the RNR, the combination of which resulting in a CTF adequate over a wide cycle range. For example, an adequate choice of y1 and y2 is to choose y1 and y2 such that y1>y2 as illustrated in FIG. 4.

The resultant CTF is illustrated in FIG. 4 as well as the quantitation cycle numbers C_{q} which are thus detected for the growth curves 200.1 and 200. 2.

FIG. 5 is illustrative of the window which is output on the display 170 (cf. FIG. 1) with a CTF that has a negative slope for the detection of c_{q1} and c_{q2} similar to the embodiment of FIG. 3.

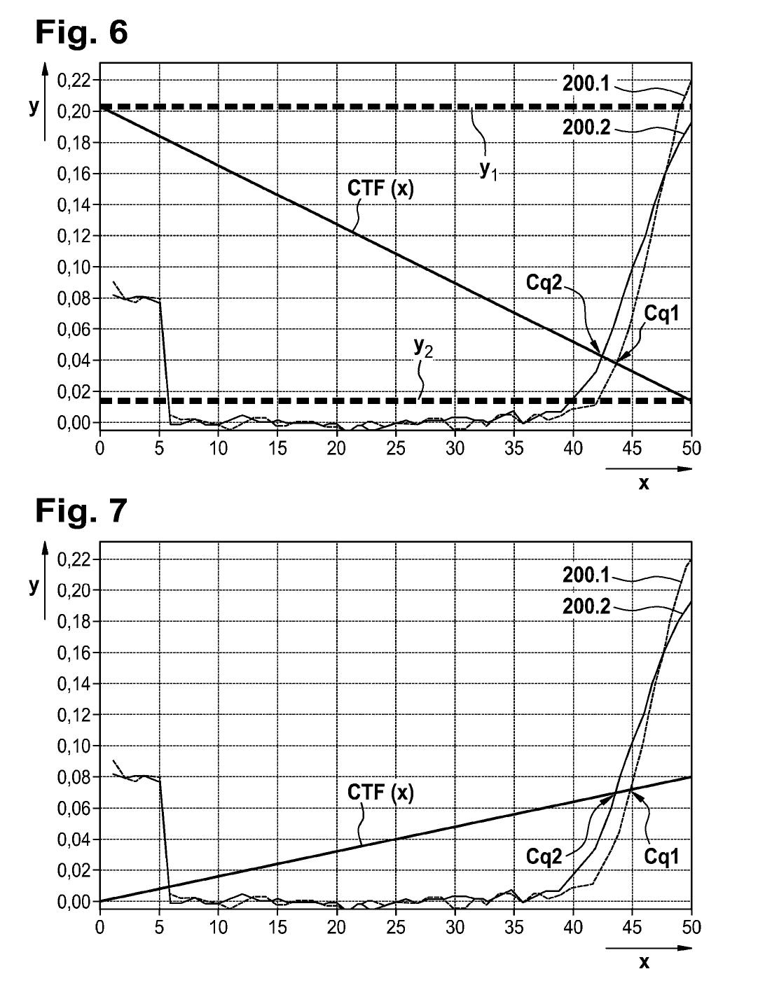

FIG. 6 illustrates a further embodiment similar to the embodiments of FIG. 3A and FIG. 3B, where the initial threshold level y1—being the RII in accordance with above equation 1—linearly transitioning in accordance with above equation 5 to the final threshold level y2—being the PGT in accordance with above equation 2.

FIG. 7 is illustrative of an embodiment where the CTF has a positive slope.

FIG. 8 illustrates the transition of the contributions of thresholds y1 and y2 to CTF for a linear, quadratic (p=2) and exponential transition in accordance with equations 5, 6 and 7, respectively.

FIG. 9 shows growth curves 200.1 to 200.7 for a dilution series where the growth curve 200.1 is obtained for the highest concentration of the analyte and the consecutive growth curves 200.2, 200.3 etc are obtained for decreasing concentrations of the analyte where the analyte concentration is decreased by one order of magnitude from one growth curve to the next, which illustrates the dynamic range of the analyzer (cf. RNR system 100 of FIG. 1).

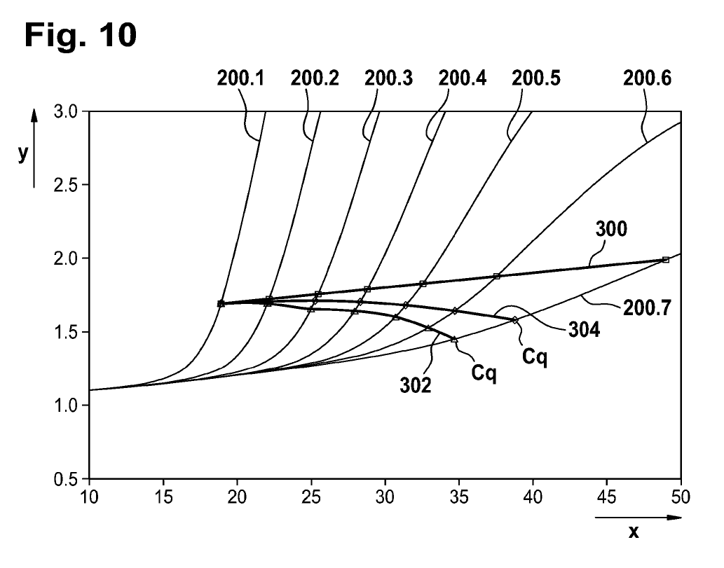

FIG. 10 is illustrative of the c_{q}-values obtained from growth curves 200.1 to 200.7 for three cases:
i. the curve 300 shows the c_{q} values that are obtained when an RII alone is used with r=50% in accordance with equation 1.
ii. the curve 302 shows the c_{q} values that are obtained if a PGT alone is used with p=0.1 in accordance with equation 2.
iii. Curve 304 is obtained when a CTF is used that combines these RII and PGT, e.g. in accordance with equation 5, 6 or 7, resulting in approximately equidistant c_{q} values for the dilution series.

As it can be observed in FIG. 10, the use of a standard CT method with a fixed threshold height, e.g RII 0.5 leads to a relative C_{q} delay for low concentration curves because of the less steep increase. On the other hand the use of a growth related CT method like PGT might over-compensate the effect of less prominent growth by early detection of the C_{q} values.

The equidistant C_{q} values that are obtained in the above case iii. reflect the constant relative concentration of the respective analytes corresponding to consequent growth curves 200.1 to 200.7 (i.e. one order of magnitude for consecutive curves) which is due to the fact that the point of intersection of the CTF with the respective growth curves is always within the exponential growth phase irrespective of the degree of dilution of the analyte over an extremely broad concentration range. This in turn implies that the precision of the C_{q} determination is substantially increased in comparison to above cases i. and ii. due to the fact that the growth curve has the lowest amount of noise within the exponential growth phase and thus yields most accurate results.

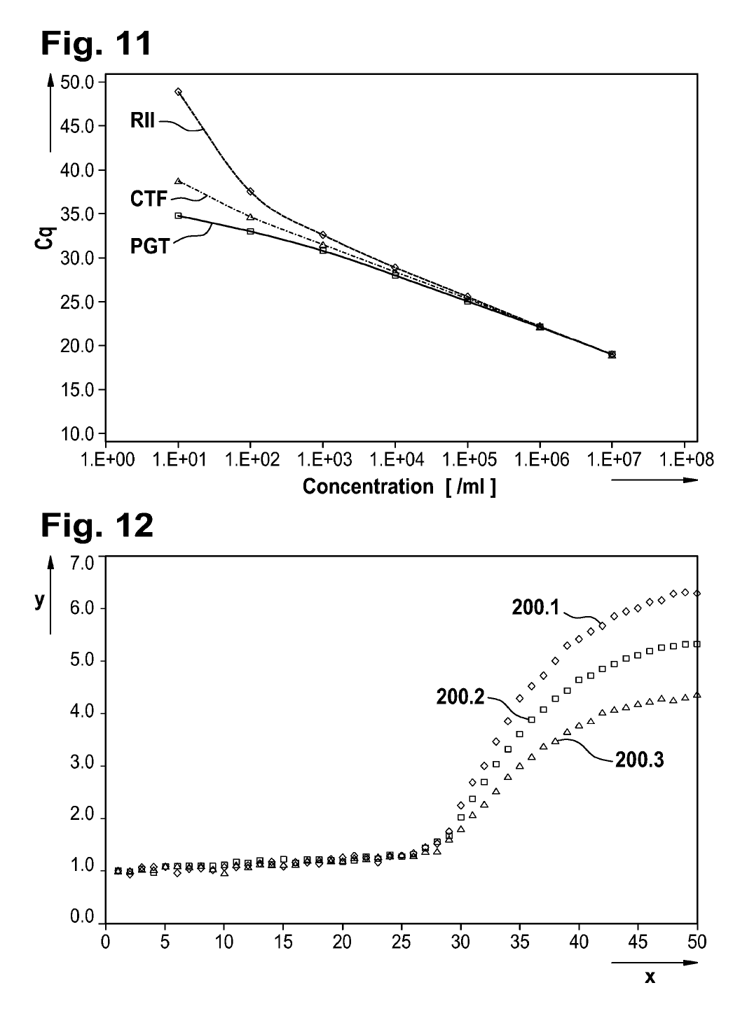

FIG. 11 shows calibration curves of the data of FIG. 10 if the RII or the PGT is used alone as well as for the combined CTF curve that provides an almost perfect linear calibration curve as apparent from FIG. 11. If a single threshold method such as RII or PGT alone is used there is a significant deviation of the calibration curve from the theoretic linear law that relates the C_{q} value to the initial analyte concentration of the sample on a logarithmic scale as apparent from FIG. 11.

FIG. 12 shows three growth curves 200.1, 200.2 and 200.3 for the same concentration of the analyte but different concentrations of an interference substance that affects the RNR reaction.

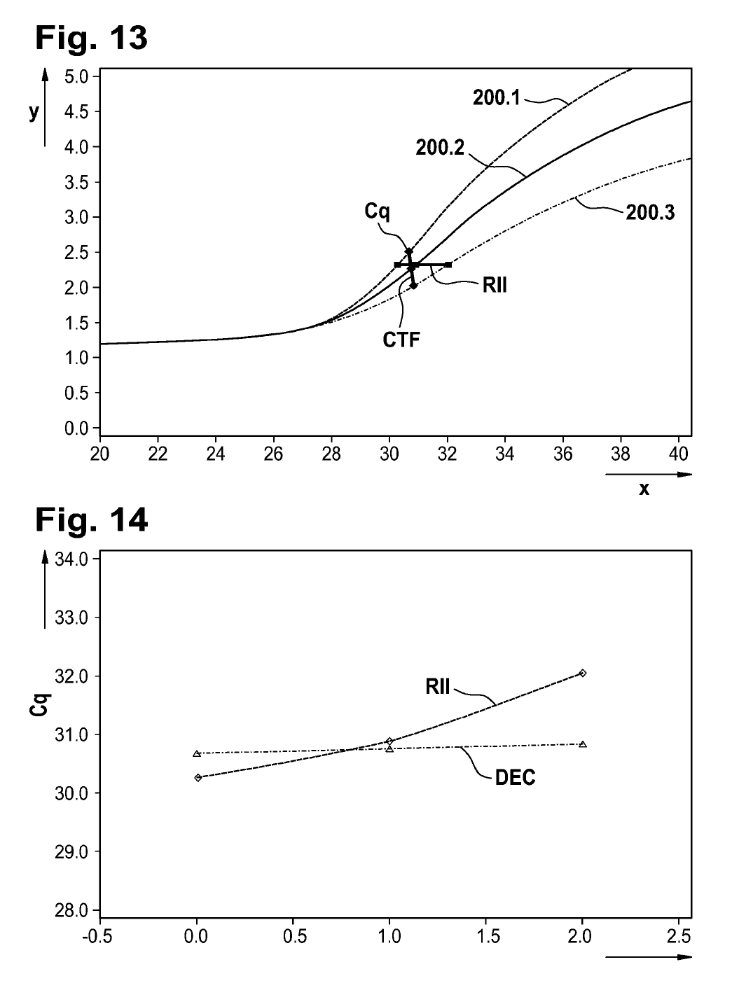

FIG. 13 shows the resultant c_{q} values that are obtained for the growth curves 200.1 to 200.3 of FIG. 12 if a flat threshold method RII is used and when a CTF with a negative slope is used such as in accordance with equations 5, 6 or 7.

As apparent from FIG. 13, the C_{q} values obtained for the three growth curves 200.1, 200.2 and 200.3 vary between 30 and 32 if the threshold method RII alone is used resulting in a respective large error. In contrast, as also illustrated in FIG. 13, the C_{q} values obtained for these curves using the negatively sloped CTF results in almost identical C_{q} values for all three curves thus greatly improving the precision even if various concentrations of interference substances are present in the sample. This is also illustrated in FIG. 14 which shows the c_{q} values of identical sample concentrations as a function of interferent concentration.

While the foregoing embodiments have been described in some detail for purposes of clarity and understanding, it will be clear to one skilled in the art from a reading of this disclosure that various changes in form and detail can be made without departing from the true scope of the subject matter. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed:

1. An analysis method for a real-time nucleic acid amplification reaction (RNR), the method comprising:
acquiring an RNR dataset representing an intensity of a fluorescence emission of an analyte over a range of reaction cycles of the RNR (RNR cycle range), each dataset including a plurality of data points each having a pair of coordinate values comprising emission signal intensity and cycle number;
creating a growth curve based on said RNR dataset indicative of the intensity of the fluorescence emission over a range of reaction cycles of the RNR (RNR cycle range);
determining a combined threshold function (CTF) over the RNR cycle range comprising at least two different threshold levels; and
identifying a Cq of the RNR, the Cq being indicative of (i) a cycle number corresponding to an intersection of the growth curve with the CTF, and (ii) a concentration of said target analyte in said sample.

2. The analysis method according to claim 1, wherein the combined threshold function (CTF) comprises two or more different threshold levels based on one or more of the following threshold methods:
relative Intercept Increase above baseline RII;
partial Growth above baseline Threshold PGT;
a multiple of standard deviation above baseline;
manually set threshold level; and
constant threshold level.

3. The analysis method according to claim 1, wherein the combined threshold function (CTF) is dependent on the cycle number of the RNR, the combined threshold function (CTF) transitioning from an initial threshold level (y1) to a final threshold level (y2), the initial threshold level (y1) being different than the final threshold level (y2).

4. The analysis method according to claim 3, wherein the transitioning of the combined threshold function (CTF) from the initial threshold level (y1) to the final threshold level (y2) is:
a linear transition;
a polynomial transition;
an exponential transition;
a step or multiple-step transition; or
a combination thereof.

5. The analysis method according to claim 3, wherein:
the initial threshold level (y1) comprises a first combination (A) of at least two different threshold levels; or
the final threshold level (y2) comprises a second combination (B) of at least two different threshold levels.

6. The analysis method according to claim 5, wherein the first combination (A) is a different combination of at least two different threshold levels than the second combination (B) of at least two different threshold levels.

7. The analysis method according to one of the claim 3, wherein:
the initial threshold level (y1) comprises at least one method adequate for early reaction cycles of the RNR; or
the final threshold level (y2) comprises at least one method adequate for late reaction cycles of the RNR.

8. The analysis method according to claim 3, wherein:
the initial threshold level (y1) has a starting point (T₁) with an y-coordinate below an y-coordinate of an end point (T₂) of the final threshold level (y2) resulting an increasing combined threshold function (CTF); or
the initial threshold level (y1) has a starting point (T₁) with an y-coordinate above an y-coordinate of an end point (T₂) of the final threshold level (y2) resulting a decreasing combined threshold function (CTF).

9. The analysis method according to claim 3, wherein the initial threshold level (y1) or the final threshold level (y2) is set at 0.

10. The analysis method according to claim 1, wherein the combination of at least two different threshold levels is constant over the RNR cycle range and the CTF comprises two or more different threshold levels based on two or more of the following methods:
relative Intercept Increase above baseline RII;
partial Growth above baseline Threshold PGT;
a multiple of standard deviation above baseline;
manually set threshold level;
constant threshold level.

11. The analysis method according to claim 10, wherein the combination comprises at least one threshold level adequate for early reaction cycles of the RNR and at least one threshold level adequate for late reaction cycles of the RNR.

12. The analysis method according to claim 5, wherein the combination is:
a linear combination of at least two different threshold levels;
a logarithmic combination of at least two different threshold levels,
or a combination thereof.

13. The analysis method according to claim 1, wherein the real-time nucleic acid amplification reaction RNR is a real-time polymerase chain reaction PCR.
